(19) United States
(12) Patent Application Publication
(10) Pub. No.: US 2024/0180601 A1
(43) Pub. Date: Jun. 6, 2024

(54) CANNULA SCREW-CABLE LOCKING SYSTEM FOR PATELLA FRACTURE

(71) Applicant: AEROSPACE CENTER HOSPITAL, Beijing (CN)

(72) Inventors: Yonghui LIANG, Beijing (CN); Junmei GAO, Beijing (CN)

(73) Assignee: AEROSPACE CENTER HOSPITAL, Beijing (CN)

(21) Appl. No.: 18/438,020

(22) Filed: Feb. 9, 2024

Related U.S. Application Data
(63) Continuation-in-part of application No. PCT/CN2022/142331, filed on Dec. 27, 2022.

(30) Foreign Application Priority Data
Mar. 17, 2022 (CN) 202210278785.3

(51) Int. Cl. A61B 17/86

(52) U.S. Cl. CPC A61B 17/8605; A61B 2017/8655

(57) ABSTRACT

A cannulated screw-cable locking system for patella fracture is provided, relating to the technology field of medical devices. The cannulated screw-cable locking system for patella fracture includes a cannulated screw, a fastening screw, and a traction cable. A head end of the cannulated screw is provided with internal threads, and a tail end of the cannulated screw is provided with external threads. The tail end of the fastening screw is threadedly connected to the cannulated screw through the internal threads. The head end of the fastening screw is fixedly connected to the traction cable.

### CROSS-REFERENCE TO RELATED APPLICATIONS

The present disclosure is a continuation-in-part application of the international patent application with the filling No. PCT/cn2022/142331, filed on Dec. 27, 2022, and claims the priority to the Chinese patent application with the filling No. 202210278785.3 filed with the Chinese Patent Office on Mar. 17, 2022, and entitled “Cannulated Screw-Cable Locking System for Patella Fracture”, the contents of which are incorporated herein by reference in entirety.

### TECHNICAL FIELD

The present disclosure relates to the technical field of medical devices and, specifically, to a cannulated screw-cable locking system for patella fracture.

### BACKGROUND ART

Currently, various fixing methods for patella fracture can be adopted, including suturing and fixation around the patella, patella fixation, fixation by AO tension band, internal fixation by the patellar concentrator, partial or complete excision of the patella, Cable-pin system, and the like. With the continuous advancement of orthopedic internal fixation instruments, the Cable-pin system has gradually been applied in fracture treatment by comprehensively utilizing the tension bands and the tension screws, and principle of converge and fixation by purse-string cerclage.

A half-thread cancellous bone compression screw and a bone tunnel guide pin are used respectively at two ends of this system. The screw, upon entering the sclerotin, exerts compression function, and the flexible steel cable is easily tightened, thus effectively preventing the screw from backing out. As this system possesses biomechanical material characteristics, the steel cable, after cerclage, exerts dynamic compression function of the tension band on the fractured ends of the patella fracture, so that the cable and the screw are integrated, thereby making the screw more secure and preventing further sliding of the steel cable. The cable and the screw interact with each other, which significantly improves the stability. However, as for the Cable-pin system, due to the screws being solid, it is necessary to ream the hole with a hollow drill and withdraw the guide pin after reduction. When inserting the screws again, the displacement of the reduction of fracture and failure to locate the screw placing tunnel can occur, resulting in loss of the limited cerclage and failure of fixation. Additionally, due to the inability to use a hollow guide pin for guidance, the open reduction is required even for a simple patellar fracture, which is not conducive to early recovery.

### SUMMARY

The objective of the present disclosure, for example, includes providing a cannulated screw-cable locking system for patella fracture, which aims at addressing at least the technical problems mentioned above.

The objective of the present disclosure, for example, can be achieved through the cannulated screw-cable locking system for patella fracture provided by the embodiments of the present disclosure. The cannulated screw-cable locking system for patella fracture includes a cannulated screw, a fastening screw, and a traction cable, wherein a head end of the cannulated screw is provided with internal threads, and a tail end of the cannulated screw is provided with external threads; a tail end of the fastening screw is threadedly connected to the cannulated screw through the internal threads; and a head end of the fastening screw is fixedly connected to the traction cable.

### BRIEF DESCRIPTION OF DRAWINGS

In order to more clearly illustrate the technical solutions of the embodiments of the present disclosure, the following will briefly introduce the drawings used in the embodiments. It should be understood that the following drawings only show some embodiments of the present disclosure, and therefore it should not be regarded as a limitation on the scope. Those ordinary skilled in the art can also obtain other related drawings based on these drawings without inventive effort.

FIG. 1 is a schematic diagram of the overall structure of the cannulated screw-cable locking system for patella fracture provided in the embodiments of the present disclosure;

FIG. 2 is a sectional view of FIG. 1 in the A-A direction;

FIG. 3 is a schematic diagram of the structure of the cannulated screw-cable locking system for patella fracture after removing the cannulated screw provided in the embodiments of the present disclosure;

FIG. 4 is a schematic structural diagram of a head end of the cannulated screw in the cannulated screw-cable locking system for patella fracture provided in the embodiments of the present disclosure;

FIG. 5 is another schematic diagram of the structure of the cannulated screw-cable locking system for patella fracture provided in the embodiments of the present disclosure;

FIG. 6 is a schematic structural diagram of a connection position between the cannulated screw and the fastening screw in FIG. 5; and

FIG. 7 is a schematic diagram of the structure of the cannulated screw in FIG. 5, without the fastening screw and traction cable.

Reference numerals: 100—cannulated screw; 110—positioning slot; 120—spline groove; 130—universal locking hole; 200—fastening screw; 300—traction cable; 400—operating hook.

### DETAILED DESCRIPTION OF EMBODIMENTS

In order to make the objective, technical solution, and advantages of the present disclosure clearer, the following will provide a clear and complete description of the technical solution in the embodiments of the present disclosure, in conjunction with the drawings in the embodiments of the present disclosure. Obviously, the described embodiments are only a part of the embodiments of the present disclosure, rather than all embodiments. Typically, the components of the embodiments of the present disclosure described and labeled in the drawings can be arranged and designed in various configurations.

Accordingly, the following detailed description of the embodiments of the present disclosure provided in the drawings is not intended to limit the scope of the claimed disclosure but merely represents selected embodiments of the present disclosure. Based on the embodiments of the present disclosure, all other embodiments obtained by those skilled in the art without inventive effort shall fall within the protection scope of the present disclosure.

It should be noted that similar numerals and letters denote similar terms in the following drawings so that once an item is defined in one drawing, it does not need to be further discussed in subsequent drawings.

In the description of the present disclosure, it should be noted that the terms “center”, “top”, “bottom”, “left”, “right”, “vertical”, “horizontal”, “inside”, “outside”, and so on indicate an orientation or positional relationship based on the orientation or positional relationship shown in the drawings, or the usual orientation or positional relationship of the disclosed product when used, which are intended only to facilitate and simplify the description of the present disclosure, not to indicate or imply that the device or element referred to must have a particular orientation, be constructed and operate in a particular orientation, and therefore are not to be construed as limiting the present disclosure. In addition, the terms “first”, “second”, and “third” are only used to distinguish the descriptive and are not to be construed as indicating or implying relative importance.

In addition, the terms “horizontal”, “vertical”, and “overhang” do not mean that elements are required to be absolutely horizontal or overhanging, but can be slightly inclined. For example, “horizontal” only means that its direction is more horizontal than “vertical”, and it does not mean that the structure must be completely horizontal, but can be slightly inclined.

In the description of the present disclosure, it should also be noted that unless otherwise clearly stipulated and limited, the terms “provide”, “install”, “connect”, and “connection” should be understood in a broad sense, for example, it can be a fixed connection, a detachable connection, or an integral connection; it can be a mechanical connection or an electrical connection; and it can be a direct connection, an indirect connection through an intermediary, or an internal communication between two components. Those of ordinary skill in the art can understand the meanings of the above terms in the present disclosure according to specific situations.

The embodiments of the present disclosure provide a cannulated screw-cable locking system for patella fracture. The cannulated screw-cable locking system for patella fracture includes a cannulated screw, a fastening screw, and a traction cable. A head end of the cannulated screw is provided with internal threads, and a tail end of the cannulated screw is provided with external threads. The tail end of the fastening screw is threadedly connected to the cannulated screw through the internal threads. The head end of the fastening screw is fixedly connected to the traction cable.

When the above-mentioned cannulated screw-cable locking system for patella fracture is used, due to the arrangement of the cannulated screw, the guide pin can be inserted through the internal through-cavity of the cannulated screw before placing the traction cable. After reduction, it is not necessary to ream the hole by the hollow drill, or insert the screws again to cause the displacement of the reduction of the fracture. It is also possible to avoid the loss of the limited sclerotin caused by the reinsertion and eliminate the need for open reduction, thereby promoting the early recovery after patella surgery. After withdrawing the guide pin, a stable connection between the traction cable and the cannulated screw is achieved by the fastening screw, further enhancing the success rate and effectiveness of the surgery.

In some embodiments, the head end of the fastening screw is integrally molded with the traction cable, which includes the following technical effects. The integrally molded design allows the traction cable and the fastening screw to form an integral structure. After mounting the fastening screw into the cannulated screw, it is possible to prevent the traction cable from loosening or slipping. The interaction between the screw and the cable significantly enhances the stability.

In some embodiments, the head end of the fastening screw is of a straight prism, and the traction cable is fixedly connected to the end face of the straight prism, which includes, for example, the following technical effects. The outline of straight prism facilitates the use of tools to fixedly screw the fastening screw into the cannulated screw, and also facilitates the replacement for other components in the event of damage to the cannulated screw, the fastening screw, or the traction cable.

In some embodiments, the traction cable is made by multiple steel cables wound in a spiral fashion, which includes, for example, the following technical effects. The structure that multiple steel cables are wound in a spiral fashion can prevent loosening or separation between the steel cables. A structure in which a steel cable with a large sectional area is combined by multiple steel cables with smaller sectional areas can enhance the flexibility of the traction cable.

In some embodiments, an end face of the head end of the cannulated screw is provided with a positioning slot. The positioning slot extends outwardly in a radial direction of the cannulated screw from the inner cavity of the cannulated screw, which includes, for example, the following technical effects. The purpose of the positioning slot is to accommodate the traction cable in the positioning slot after the traction cable has been fixed in surgery, thereby preventing the traction cable from protruding beyond the cannulated screw to cause damage to other tissues or organs.

In some embodiments, multiple positioning slots are provided, wherein the multiple positioning slots are distributed at an equal interval around the cannulated screw in a circumferential direction, which includes, for example, the following technical effects. The structure of providing multiple positioning slots allows the traction cable to be wound and fixed in different directions, thereby enhancing the flexibility in the use of equipment.

In some embodiments, the end face of the head end of the cannulated screw is provided with a spline groove, and the inner cavity of the cannulated screw is communicated with the groove bottom of the spline groove, which includes, for example, the following technical effects. The spline groove facilitates the use of tools to screw the cannulated screw into the patella, thus completing the initial operation of the surgery.

In some embodiments, a radial dimension of the head end of the cannulated screw is greater than a diameter of the cannulated screw, which includes, for example, the following technical effects. The larger radial dimension at the head end can effectively provide a compression effect.

In some embodiments, an operating hook is provided, wherein the operating hook is fixedly connected to a free end of the traction cable, which includes, for example, the following technical effects. The operating hook is configured to insert to make the traction cable, along the outline of the patella, pass through the human tissue.

In some embodiments, the operating hook can be curved, which includes, for example, the following technical effects. The curved shape of the operating hook fits better to the outer surface of the patella, thus facilitating a smoother threading operation.

In some embodiments, a side wall of the fastening screw is provided with an elastic pin and an inner wall of the cannulated screw is provided with a pinhole. After inserting the fastening screw into the cannulated screw, self-locking fixation between the fastening screw and the cannulated screw is achieved.

In some embodiments, the positioning slot is provided as an arc-shaped slot to attach and connect with the traction cable.

In some embodiments, the arc-shaped bending angle of the operating hook is set at 30 degrees.

In some embodiments, a screw tail of the cannulated screw is in an internal rectangular or hexagonal shape.

In some embodiments, the traction cable is a metallic steel cable or a cable made of a composite material.

In some embodiments, a universal locking hole is provided inside the head end of the cannulated screw. The tail end of the fastening screw extends, in the state of being screwed to the cannulated screw, obliquely relatively to a longitudinal axis of the cannulated screw from the universal locking hole, which includes, for example, the following technical effects. The universal locking hole is able to ensure that the traction cable is fixed at a specific traction angle, and the fastening screw, extending obliquely, provides a better fixation effect on the patella, especially in case of the fissure of inferior pole of patella or comminuted patella fracture.

In some embodiments, a center axis of the universal locking hole is inclined at an angle within the range of 0-45 degrees relative to the longitudinal axis of the cannulated screw.

In some embodiments, multiple universal locking holes can be provided. The multiple universal locking holes (130) are distributed at an equal interval around the cannulated screw in the circumferential direction and the angles of inclination of the multiple universal locking holes relative to the longitudinal axis of the cannulated screw are different from each other.

In some embodiments, the cannulated screws and the fastening screws are provided in an even number, and the traction cables are correspondingly connected in pairs at their free ends.

In some embodiments, two cannulated screws and two fastening screws are provided.

Some embodiments of the present disclosure are described in detail below, in conjunction with the drawings. In situations where they do not conflict, the features described in the following embodiments and their characteristics can be combined with each other.

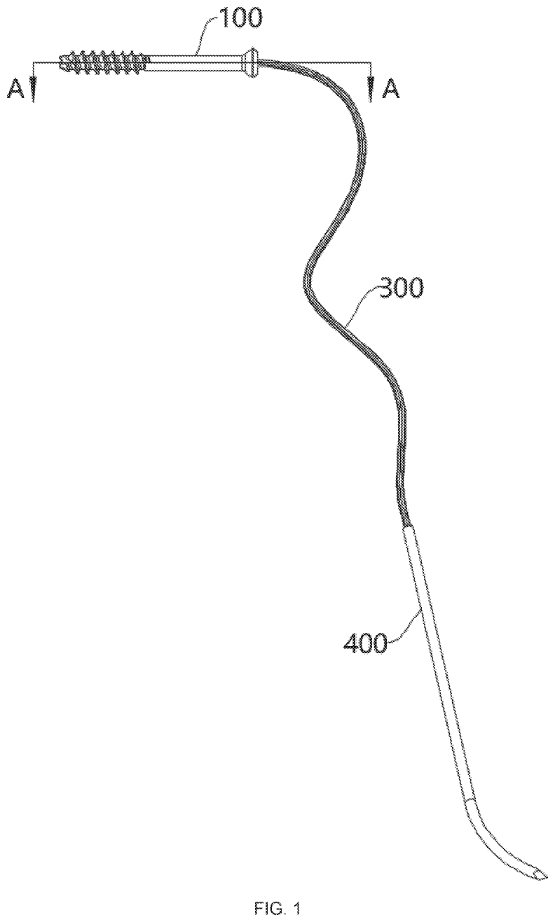

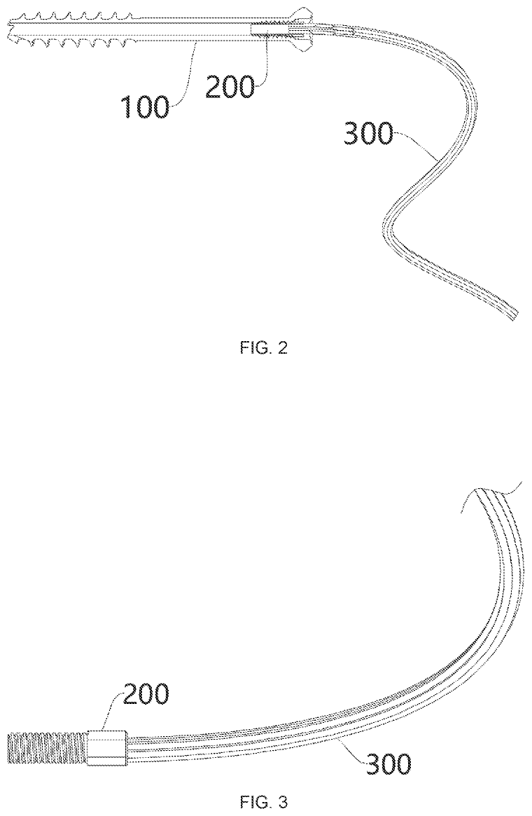

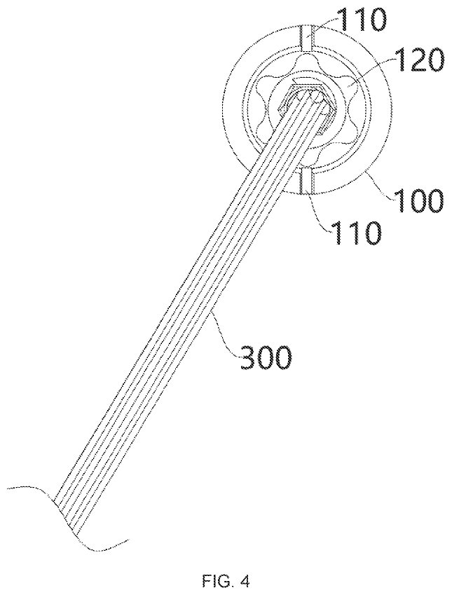

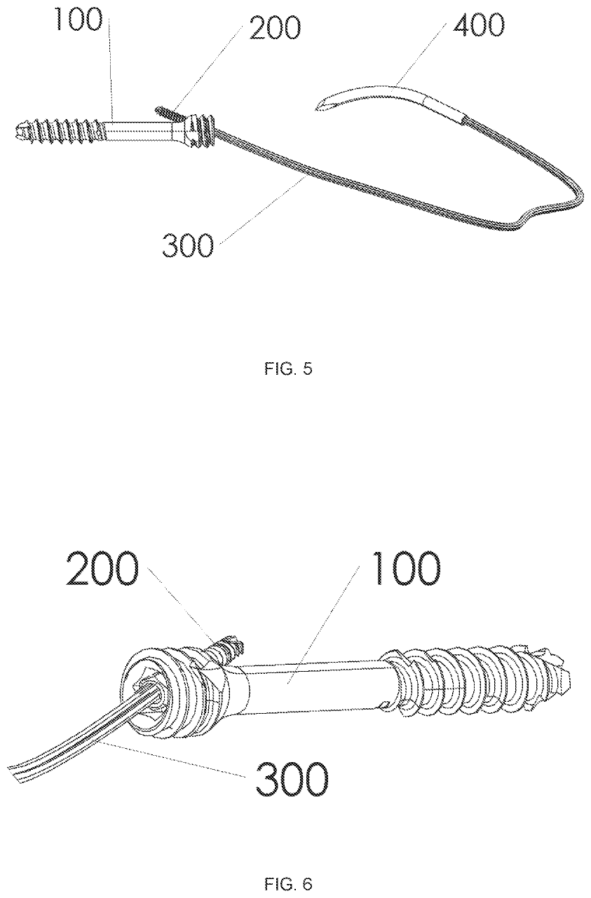

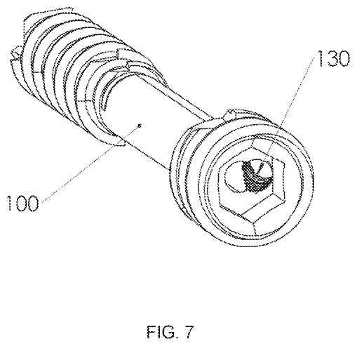

FIG. 1 is a schematic diagram of the overall structure of a cannulated screw-cable locking system for patella fracture provided in the embodiments of the present disclosure; FIG. 2 is a sectional view of FIG. 1 in the A-A direction; FIG. 3 is a schematic diagram of the structure of the cannulated screw-cable locking system for patella fracture after removing the cannulated screw 100 provided in the embodiments of the present disclosure; FIG. 4 is a schematic structural diagram of a head end of the cannulated screw 100 in the cannulated screw-cable locking system for patella fracture provided in the embodiments of the present disclosure; FIG. 5 is another schematic diagram of the structure of the cannulated screw-cable locking system for patella fracture provided in the embodiments of the present disclosure; FIG. 6 is a schematic structural diagram of a connection position between the cannulated screw and the fastening screw in FIG. 5; and FIG. 7 is a schematic diagram of the structure of the cannulated screw in FIG. 5, in which the fastening screw and traction cable are omitted. Referring to FIGS. 1 to 5, the embodiments of the present disclosure provide a cannulated screw-cable locking system for patella fracture, including a cannulated screw 100, a fastening screw 200, and a traction cable 300.

A head end of the cannulated screw 100 can be provided with internal threads, and a tail end of the cannulated screw 100 can be provided with external threads. The tail end of the fastening screw 200 can be threadedly connected to the cannulated screw 100 through the internal threads. The head end of the fastening screw 200 can be fixedly connected to the traction cable 300.

In the above structure, the cannulated screw 100 not only functions to be inserted into the sclerotin for fixation, but also, due to the arrangement of the hollow through-cavity, achieves a guiding and introducing function. The fastening screw 200 functions to fix the traction cable 300 to the cannulated screw 100.

The cannulated screw 100 can be provided with threads only at the tail end. A screw tail of the cannulated screw 100 can be in an internal rectangular or hexagonal shape.

The traction cable can be selected from either a metallic steel cable or a cable made of a composite material.

The fastening screw 200 can be connected to the cannulated screw 100 through the threads. Optionally, as for the fastening screw 200, a self-locking structure can also be utilized to achieve a locking connection with the cannulated screw 100. For example, a side wall of the fastening screw 200 can be provided with an elastic pin and an inner wall of the cannulated screw 100 can be provided with a pinhole. After inserting the fastening screw 200 into the cannulated screw 100, self-locking fixation can be achieved.

The cannulated screw 100 and the fastening screw 200 can rotate in the same direction or in opposite directions. In the optional embodiment, their rotation directions are set to be opposite, which means that one of the thread of the cannulated screw 100 and the thread of the fastening screw 200 can be left-hand thread and the other one can be right-hand thread.

The working principle and operating method of the cannulated screw-cable locking system for patella fracture in the embodiments of the present disclosure are as follows.

The cannulated screw-cable locking system for patella fracture, due to the arrangement of the cannulated screw 100, is able to allow the insertion of the guide pin through the internal through-cavity of the cannulated screw 100 before placing the traction cable 300. After reduction, there is no need to ream the hole by hollow drill, or insert the screws again, which leads to the displacement of the reduction of the fracture. It is also possible to avoid the loss of the limited sclerotin caused by the second insertion and eliminate the need for open reduction, thereby promoting early recovery after patella surgery. After withdrawing the guide pin, a stable connection between the traction cable 300 and the cannulated screw 100 can be achieved by the fastening screw 200, further enhancing the success rate and effectiveness of the surgery.

In at least one optional embodiment, as shown in FIG. 2, FIG. 3, and FIG. 6, the head end of the fastening screw 200 can be integrally molded with the traction cable 300. In the design of the embodiment, the integrally molded design allows the traction cable 300 and the fastening screw 200 to form an integral structure. After mounting the fastening screw 200 into the cannulated screw 100, it is possible to prevent the traction cable 300 from loosening or slipping. The interaction between the screw and the cable significantly enhances the stability.

Optionally, the traction cable 300 can also be fixedly connected to the fastening screw 200 in a welding or clamping manner.

In at least one optional embodiment, as shown in FIG. 3, the head end of the fastening screw 200 can be of a straight prism, and the traction cable 300 can be fixedly connected to the end face of the straight prism. In the design of the embodiment, the outline of straight prism facilitates the use of tools to fixedly screw the fastening screw 200 into the cannulated screw 100, and also facilitates the replacement for other components in the event of damage to the cannulated screw 100, the fastening screw 200, or the traction cable 300.

The head end of the fastening screw 200 can be of a triangular prism, quadrangular prism, or hexagonal prism so as to facilitate the use of tools for screwing and installation after insertion.

In at least one optional embodiment, as shown in FIGS. 1 to 5, the traction cable 300 can be made by multiple steel cables wound in a spiral fashion. In the design of the embodiment, the structure that the multiple steel cables are wound in a spiral fashion prevents loosening and separation between the steel cables. A structure in which a steel cable with a large sectional area is combined by multiple steel cables with smaller sectional areas enhances the flexibility of the traction cable 300.

The traction cable 300 can also be made from a single steel cable so as to be integrally molded with the fastening screw 200.

In at least one optional embodiment, as shown in FIG. 4, an end face of the head end of the cannulated screw 100 can be provided with a positioning slot 110. The positioning slot 110 can extend outwardly in a radial direction of the cannulated screw 100 from the inner cavity of the cannulated screw 100. In the design of the embodiment, for example, the purpose of the positioning slot 110 includes accommodating the traction cable 300 in the positioning slot 110 after the traction cable 300 has been surgically fixed, thus preventing the traction cable 300 from protruding beyond the cannulated screw 100 to cause damage to other tissues or organs.

In at least one optional embodiment, as shown in FIG. 4, the number of positioning slots 110 can be multiple, wherein the multiple positioning slots 110 can be distributed at an equal interval around the cannulated screw 100 in a circumferential direction. In the design of the embodiment, the structure of providing multiple positioning slots 110 can allow the traction cable 300 to be wound and fixed in different directions, thereby enhancing flexibility in the use of equipment.

The positioning slots 110 can be provided in a number of two, three, four, or more. Moreover, the positioning slot 110 should be provided as an arc-shaped slot to facilitate attaching and connecting to the traction cable 300, thus preventing deformation of the traction cable 300 from reducing tensile stress.

In at least one optional embodiment, as shown in FIG. 4, the end face of the head end of the cannulated screw 100 can be provided with a spline groove 120, and the inner cavity of the cannulated screw 100 can be communicated with the groove bottom of the spline groove 120. In the design of the embodiment, the spline groove 120 facilitates the use of tools to screw the cannulated screw 100 into the patella, thus completing the initial operation of the surgery.

In at least one optional embodiment, as shown in FIG. 1, FIG. 2, FIG. 4, and FIG. 5, a radial dimension of the head end of the cannulated screw 100 can be greater than a diameter of the cannulated screw 100. In the design of the embodiment, the larger radial dimension at the head end can effectively provide a compression effect.

In at least one optional embodiment, as shown in FIG. 1 and FIG. 5, the cannulated screw-cable locking system for patella fracture can also include an operating hook 400, wherein the operating hook 400 can be fixedly connected to a free end of the traction cable 300. In the design of the embodiment, the operating hook 400 is inserted to make the traction cable 300, along the outline of the patella, pass through the human tissue. In at least one optional embodiment, as shown in FIG. 1 and FIG. 5, the operating hook 400 can be curved. In the design of the embodiment, the curved shape of the operating hook 400 fits better to the outer surface of the patella, thus facilitating a smooth threading operation.

Optionally, the arc-shaped bending angle of the operating hook 400 can be set at 30 degrees.

In at least one optional embodiment, as shown in FIGS. 5 to 7, the head end of the cannulated screw 100 is also provided with external threads so as to enable the cannulated screw to enter the sclerotin more easily.

In at least one optional embodiment, as shown in FIG. 5 and FIG. 6, the tail end of the fastening screw 200 extends, in the state of being screwed to the cannulated screw 100, obliquely relatively to a longitudinal axis of the cannulated screw 100 from the universal locking hole 130. As shown in FIG. 7, the universal locking hole 130 is arranged inside the head end of the cannulated screw 100 and has a certain inclined angle relative to the longitudinal axis of the cannulated screw. Optionally, the inclined angle is within the range of 0-45 degrees, for example, 10 degrees, 15 degrees, 25 degrees, 30 degrees, and 45 degrees. The universal locking hole 130 provides a larger operable angle for the insertion of the fastening screw 200 so as to enable the traction cable 300 to be fixed at a specific traction angle.

Optionally, the number of universal locking holes 130 can be multiple, for example, two or three. Multiple universal locking holes 130 can be distributed at an equal interval around the cannulated screw 100 in the circumferential direction, and the inclined angles of the multiple universal locking holes 130 relative to the longitudinal axis of the cannulated screw can be different from each other, such that a specific traction angle can be selected as required.

In at least one optional embodiment, in the cannulated screw-cable locking system for patella fracture, the number of the cannulated screw 100 and the fastening screw 200 is an even number, for example, two; and the traction cables 300 are correspondingly connected in pairs at their free ends. When using two cannulated screws 100 for fixation, the traction cables 300 connecting the two fastening screws 200 cross in the shape of the Chinese character “ [symbol] ”, thus achieving lateral fixation. At this point, the two cannulated screws 100 can pull each other through the traction cables 300, thus completing compression fixation for the patella. The traction cables are tightened and fixed using a binder. Depending on the direction of the bone, a 270-degree covering can be achieved.

The results of finite element analysis for the cannulated screw-cable locking system for patella fracture according to the present disclosure in different scenarios (common fracture and comminuted fracture) are shown in the tables below, with the control group being the Kirschner wire-tension band system and the Cable-Pin system.

TABLE 1
(common fracture, with distance between screw and anterior
patella being 5 mm, load of 400&800N, and direction of 0°)

| | 0°-400N-5 mm | | 0°-800N-5 mm | |
|---|---|---|---|---|
| | patella | fracture | patella | fracture |
| | maximum | surface | maximum | surface |
| | total | maximum | total | maximum |
| | displacement | gap | displacement | gap |
| model | (mm) | (mm) | (mm) | (mm) |
| Kirschner | 4.5982 | 0.42671 | 9.431 | 0.71317 |
| wire-tension | | | | |
| band | | | | |
| Cabel-Pin | 4.2653 | 0.48067 | 8.4102 | 0.6771 |
| cannulated | 3.7857 | 0.37271 | 7.4711 | 0.62323 |
| screw-cable | | | | |
| locking | | | | |
| system | | | | |
| according to | | | | |
| the present | | | | |
| disclosure | | | | |

TABLE 2
(common fracture, with distance between screw and anterior
patella being 10 mm, load of 400&800N, and direction of 0°)

| | 0°-400N-10 mm | | 0°-800N-10 mm | |
|---|---|---|---|---|
| | patella | fracture | patella | fracture |
| | maximum | surface | maximum | surface |
| | total | maximum | total | maximum |
| | displacement | gap | displacement | gap |
| model | (mm) | (mm) | (mm) | (mm) |
| Kirschner | 3.7546 | 0.42183 | 7.3328 | 0.70354 |
| wire-tension | | | | |
| band | | | | |
| Cabel-Pin | 3.1864 | 0.26605 | 6.2077 | 0.41587 |
| cannulated | 2.947 | 0.14473 | 5.9065 | 0.26903 |
| screw-cable | | | | |
| locking | | | | |
| system | | | | |
| according to | | | | |
| the present | | | | |
| disclosure | | | | |

TABLE 3
(common fracture, with distance between screw and anterior
patella being 5 mm, load of 400&800N, and direction of 45°)

| | 45°-400N-5 mm | | 45°-800N-5 mm | |
|---|---|---|---|---|
| | patella | fracture | patella | fracture |
| | maximum | surface | maximum | surface |
| | total | maximum | total | maximum |
| | displacement | gap | displacement | gap |
| model | (mm) | (mm) | (mm) | (mm) |
| Kirschner | 0.42826 | 0.30496 | 0.72025 | 0.47696 |
| wire-tension | | | | |
| band | | | | |
| Cabel-Pin | 0.27162 | 0.13634 | 0.50439 | 0.21927 |
| cannulated | 0.21577 | 0.054843 | 0.42405 | 0.10294 |
| screw-cable | | | | |
| locking | | | | |
| system | | | | |
| according to | | | | |
| the present | | | | |
| disclosure | | | | |

TABLE 4
(common fracture, with distance between screw and anterior
patella being 10 mm, load of 400&800N, and direction of 45°)

| | 45°-400N-5 mm | | 45°-800N-5 mm | |
|---|---|---|---|---|
| | patella | fracture | patella | fracture |
| | maximum | surface | maximum | surface |
| | total | maximum | total | maximum |
| | displacement | gap | displacement | gap |
| model | (mm) | (mm) | (mm) | (mm) |
| Kirschner | 0.42445 | 0.29509 | 0.76103 | 0.50058 |
| wire-tension | | | | |
| band | | | | |
| Cabel-Pin | 0.37755 | 0.25683 | 0.68162 | 0.43387 |
| cannulated | 0.30328 | 0.18019 | 0.58429 | 0.33702 |
| screw-cable | | | | |
| locking | | | | |
| system | | | | |
| according to | | | | |
| the present | | | | |
| disclosure | | | | |

TABLE 5
(comminuted fracture, with distance between screw and anterior
patella being 10 mm, load of 400&800N, and direction of 0°)

| | 0°-400N-10 mm | | 0°-800N-10 mm | |
|---|---|---|---|---|
| | patella | fracture | patella | fracture |
| | maximum | surface | maximum | surface |
| | total | maximum | total | maximum |
| | displacement | gap | displacement | gap |
| model | (mm) | (mm) | (mm) | (mm) |
| Cabel-Pin | 4.6772 | 0.70628 | 9.0043 | 0.8879 |
| cannulated | 3.767 | 0.24191 | 7.3522 | 0.28892 |
| screw-cable | | | | |
| locking | | | | |
| system | | | | |
| according to | | | | |
| the present | | | | |
| disclosure | | | | |

TABLE 6
(comminuted fracture, with distance between screw and anterior
patella being 10 mm, load of 400&800N, and direction of 0°)

| | 45°-400N-10 mm | | 45°-800N-10 mm | |
|---|---|---|---|---|
| | patella | fracture | patella | fracture |
| | maximum | surface | maximum | surface |
| | total | maximum | total | maximum |
| | displacement | gap | displacement | gap |
| model | (mm) | (mm) | (mm) | (mm) |
| Cabel-Pin | 0.41748 | 0.32683 | 0.77609 | 0.57759 |
| cannulated | 0.3186 | 0.22804 | 0.59382 | 0.29039 |
| screw-cable | | | | |
| locking | | | | |
| system | | | | |
| according to | | | | |
| the present | | | | |
| disclosure | | | | |

TABLE 7
(comminuted fracture, with distance between screw and anterior
patella being 10 mm, load of 400&800N, and direction of 90°)

| | 90°-400N-10 mm | | 90°-800N-10 mm | |
|---|---|---|---|---|
| | patella | fracture | patella | fracture |
| | maximum | surface | maximum | surface |
| | total | maximum | total | maximum |
| | displacement | gap | displacement | gap |
| model | (mm) | (mm) | (mm) | (mm) |
| Cabel-Pin | 1.1828 | 0.2193 | 2.3262 | 0.40827 |
| cannulated | 1.1579 | 0.14825 | 2.1968 | 0.26237 |
| screw-cable | | | | |
| locking | | | | |
| system | | | | |
| according to | | | | |
| the present | | | | |
| disclosure | | | | |

Based on the data in the tables, it can be concluded that, whether it is a comminuted fracture or a common fracture, under the same load conditions, the fixation effect of the cannulated screw-cable locking system according to the present disclosure is superior to that of the Kirschner wire-tension band and Cabel-Pin systems used in the prior art.

The cannulated screw-cable locking system for patella fracture described in the method embodiments is not only applicable to the patella but can also be applied, as needed, to fractures in locations such as the olecranon of the ulna, the tip of the greater trochanter of the femur, and the medial and lateral malleolus.

The above is only an optional embodiment of the present disclosure, which is not intended to limit, and the present disclosure may have various changes and variations for those skilled in the art. Any modification, equivalent substitution, improvement, etc. made within the spirit and principles of the present disclosure shall be included in the scope of protection of the present disclosure.

### INDUSTRIAL PRACTICALITY

The embodiments of the present disclosure provide a cannulated screw-cable locking system for patella fracture. The cannulated screw-cable locking system for patella fracture includes a cannulated screw, a fastening screw, and a traction cable. A head end of the cannulated screw is provided with internal threads, and a tail end of the cannulated screw is provided with external threads. The tail end of the fastening screw is threadedly connected to the cannulated screw through the internal threads. The head end of the fastening screw is fixedly connected to the traction cable. When the above-mentioned cannulated screw-cable locking system for patella fracture is used, due to the arrangement of the cannulated screw, the guide pin can be inserted through the internal through-cavity of the cannulated screw before placing the traction cable. After reduction, it is not necessary to ream the hole by the hollow drill, or insert the screws again to cause the displacement of the reduction of the fracture. It is also possible to avoid the loss of the limited sclerotin caused by the reinsertion and eliminate the need for open reduction, thereby promoting the early recovery after patella surgery. After withdrawing the guide pin, a stable connection between the traction cable and the cannulated screw is achieved by the fastening screw, further enhancing the success rate and effectiveness of the surgery.

Additionally, it is noted that the cannulated screw-cable locking system for patella fracture provided in the embodiments of the present disclosure is reproducible and applicable in various industrial applications. The cannulated screw-cable locking system for patella fracture provided in the embodiments of the present disclosure can be used in the technical field of medical devices.

What is claimed is:

1. A cannulated screw-cable locking system for patella fracture, wherein the cannulated screw-cable locking system for patella fracture comprises a cannulated screw, a fastening screw, and a traction cable, wherein a head end of the cannulated screw is provided with internal threads, and a tail end of the cannulated screw is provided with external threads; a tail end of the fastening screw is threadedly connected to the cannulated screw through the internal threads; and a head end of the fastening screw is fixedly connected to the traction cable.

2. The cannulated screw-cable locking system for patella fracture according to claim 1, wherein the head end of the fastening screw is integrally molded with the traction cable.

3. The cannulated screw-cable locking system for patella fracture according to claim 1, wherein the head end of the fastening screw is of a straight prism, and the traction cable is fixedly connected to an end face of the straight prism.

4. The cannulated screw-cable locking system for patella fracture according to claim 1, wherein the traction cable is made by multiple steel cables wound in a spiral fashion.

5. The cannulated screw-cable locking system for patella fracture according to claim 1, wherein an end face of the head end of the cannulated screw is provided with a positioning slot, and the positioning slot extends outwardly in a radial direction of the cannulated screw from an inner cavity of the cannulated screw.

6. The cannulated screw-cable locking system for patella fracture according to claim 5, wherein multiple positioning slots are provided, wherein the multiple positioning slots are distributed at an equal interval around the cannulated screw in a circumferential direction.

7. The cannulated screw-cable locking system for patella fracture according to claim 1, wherein an end face of the head end of the cannulated screw is provided with a spline groove, and an inner cavity of the cannulated screw is communicated with a groove bottom of the spline groove.

8. The cannulated screw-cable locking system for patella fracture according to claim 1, wherein a radial dimension of the head end of the cannulated screw is greater than a diameter of the cannulated screw.

9. The cannulated screw-cable locking system for patella fracture according to claim 1, wherein the cannulated screw-cable locking system for patella fracture further comprises an operating hook, wherein the operating hook is fixedly connected to a free end of the traction cable.

10. The cannulated screw-cable locking system for patella fracture according to claim 9, wherein the operating hook is curved.

11. The cannulated screw-cable locking system for patella fracture according to claim 1, wherein a side wall of the fastening screw is provided with an elastic pin and an inner wall of the cannulated screw is provided with a pinhole, and by inserting the elastic pin into the pinhole, a self-locking fixation between the fastening screw and the cannulated screw is achieved.

12. The cannulated screw-cable locking system for patella fracture according to claim 5, wherein the positioning slot is provided as an arc-shaped slot to attach and connect with the traction cable.

13. The cannulated screw-cable locking system for patella fracture according to claim 9, wherein an arc-shaped bending angle of the operating hook is set at 30 degrees.

14. The cannulated screw-cable locking system for patella fracture according to claim 1, wherein a screw tail of the cannulated screw is in an internal rectangular or hexagonal shape.

15. The cannulated screw-cable locking system for patella fracture according to claim 1, wherein the traction cable is a metallic steel cable or a cable made of a composite material.

16. The cannulated screw-cable locking system for patella fracture according to claim 1, wherein the head end of the cannulated screw is provided with external threads.

17. The cannulated screw-cable locking system for patella fracture according to claim 1, wherein a universal locking hole is provided inside the head end of the cannulated screw; and the tail end of the fastening screw extends, in a state of being screwed to the cannulated screw, obliquely relatively to a longitudinal axis of the cannulated screw from the universal locking hole.

18. The cannulated screw-cable locking system for patella fracture according to claim 16, wherein a center axis of the universal locking hole is inclined at an angle within a range of 0-45 degrees relative to a longitudinal axis of the cannulated screw.

19. The cannulated screw-cable locking system for patella fracture according to claim 16, wherein multiple universal locking holes are provided; and the multiple universal locking holes are distributed at an equal interval around the cannulated screw in a circumferential direction, and angles of inclination of the multiple universal locking holes relative to a longitudinal axis of the cannulated screw are different from each other.

20. The cannulated screw-cable locking system for patella fracture according to claim 1, wherein the number of cannulated screws and fastening screws is an even number, and traction cables are correspondingly connected in pairs at free ends of the traction cables.
